(19) United States
(12) Patent Application Publication
(10) Pub. No.: US 2007/0197917 A1
(43) Pub. Date: Aug. 23, 2007

(54) Continuous-focus ultrasound lens

(76) Inventor: Jan Peter Bagge, Stenlose (DK)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/642,654

(22) Filed: Dec. 21, 2006

Related U.S. Application Data
(60) Provisional application No. 60/752,599, filed on Dec. 22, 2005.

(51) Int. Cl. A61B 8/14

(52) U.S. Cl. 600/459

(57) ABSTRACT

The depth of focus in the elevation plane of an acoustic ultrasound transducer is extended. The ultrasound transducer comprises an acoustic element, said element having a substantially uniform frequency amplitude characteristic across its spatial extent and transmitting an ultrasound beam when excited, an acoustic lens positioned in front of said element, said lens having a cross sectional profile comprising (1) a curved portion with a curved front surface and a back surface facing said transducer element, said curved lens portion providing a focal point at a first focal range, and (2) a pair of linear portions with linear front surfaces and back surfaces facing said transducer element, said linear portions positioned on either side of said curved portion, and said linear portions providing continuous focusing at imaging ranges after said first focal depth of said curved portion. The broadband frequency characteristic of said element means that all frequencies are focused at all focal points, which makes the invention particularly useful for harmonic ultrasound imaging.

The invention is related to the field of medical ultrasound imaging, and particularly the area of improving of the elevation beam profile of ultrasound transducers to obtain better slice thickness and image contrast resolution by means of extending the depth of focus. The elevation beam profile is described by the slice thickness perpendicular to the imaging plane, often referred to the elevation beamwidth, and the sidelobe characteristics of the beam perpendicular to the imaging plane.

### BACKGROUND OF THE INVENTION

Ultrasound imaging is an established modality in the field of medical imaging providing real-time images of soft human tissue and blood flow. The images are generated using a hand held transducer comprising either a single element or most commonly an array of elements arranged in a single row (1D transducers). For array transducers, cross-sectional images are conventionally created by transmitting focused beams in multiple directions sequentially using all or a subsection of the transducer elements, receiving the echoes generated by the tissue for each direction using selected ones of the transducer elements, and processing the signals for each direction separately to form scan lines according to the transmit directions. The set of scan lines are subsequently scan converted and presented to the user on a dedicated monitor. The processing of the received echoes from each transmit direction typically involves beam forming, where the signals from the receive elements are delayed, weighted, and summed to focus the echoes along the transmit direction. Most often, the delays and weighting coefficients are updated continuously, producing dynamic focusing and apodization to obtain a desired receive beam profile.

The dynamic receive processing applied on the received echoes is typically directed towards obtaining a preferably narrow and uniform lateral beam profile providing high spatial and contrast resolution within the imaging plane.

The slice thickness perpendicular to the imaging plane, often referred to as the elevation beamwidth, affects the image contrast resolution due to the complex summation at the element surfaces of all scatters within the beam at a given time instance (or equally imaging depth). If a wide elevation beam is produced by the transducer, a small target, e.g. a small malignant lesion, may be undetectable due to the surrounding tissue scatters. Hence, a narrow and uniform slice thickness is desirable.

FIG. 1 is a cross sectional view of a transducer element typically applied in modern ultrasound probes, here a 1D transducer (not drawn to scale). The acoustic element is typically made of a piezoelectric ceramic (PZT) 12 which converts an electric excitation signals to an acoustic wave in transmission mode and vice versa in reception mode. The backing layer 10 is introduced to improve the broadband characteristics of the element and attenuate internal reflections, and the matching layer(s) 14 (only one is shown) provide impedance matching to human tissue to more effective transfer of the acoustic energy. Elevation focusing is traditionally obtained by means of an acoustic lens 16 providing a fixed focus using a circular shaped lens surface 18. The lens is typically made of silicone material, often selected from the RTV silicone family. The sound speed of the lens material applied in FIG. 1 is less than the typical sound speed of the objects for which the transducer is intended (not shown). The circular lens surface profile is obtained as a result of the sound speed of the lens material being lower than the sound speed of the intended object and the desired focal depth. The design of single focus acoustic lenses for medical transducers is a well known and well documented discipline in the prior art. The focal depth of the lens is chosen to emphasize a desirable tissue region depending on the intended application of the probe. The area around the focal point where the elevation beamwidth is approximately constant, often called the depth of focus, is, for a given focal depth, determined by the size of the elevation aperture. Decreasing the aperture produces a weaker focus resulting in a wider beamwidth larger depth of focus (lower contrast resolution over a larger imaging range) and more uniform intensity distribution. Increasing the aperture produces a sharper focus with a more narrow beamwidth at the focus, smaller depth of focus (better contrast resolution over a smaller imaging range), and higher concentration of the intensity at the focal point. Additionally, the size of the elevation aperture limits the acoustical power that can be transmitted to the object by each element, and hence the signal-to-noise ratio obtainable. Furthermore, it determines the element sensitivity in receive mode, again affecting the signal to noise ratio. Thus, the elevation aperture and lens focal depth is both a tradeoff between contrast resolution and sensitivity at the focal point and away from the focal point. For typical lenses applied in the state of the art, the ratio of focal depth to elevation aperture (often called the f-number) commonly ranges between 3 and 6, producing medium to weak focus. The slice thickness and contrast resolution of such lenses are significantly worse outside the focal region than their performance at the focal point. Dynamic receive processing in the imaging plane is performed to obtain as narrow and uniform a beamwidth as possible for optimum spatial and contrast resolution. Therefore, similar means for obtaining a narrow and preferably uniform slice thickness in the elevation plane is greatly desired.

Within the field of the invention, various methods have been developed to improve the elevation performance of transducers.

In U.S. Pat. No. 5,083,568 an acoustic bi-focal lens is disclosed, which focuses the center part of the elevation aperture at a closer range and a pair of symmetrically selected outer segments are focused at a deeper focal point. Each piezoelectric element (the ceramic) is sliced at the points of transition from the first central focusing segment to the second outer focusing segments. The central and outer segments are controlled using signal electrodes mounted on the ceramic surfaces to produce an ultrasound wave emanating from the excited segment. The outer segments are controlled together and separately from the central segment. With this invention, the central row of elements in the corresponding transducer array is used when imaging targets at shallow ranges and the outer rows are used when deeper laying structures are examined.

Hossack et. al. developed in U.S. Pat. Nos. 5,678,554 and 6,027,448 a transducer array composed of elements with frequency dependent elevation characteristics, where the ceramic (the piezoelectric material) has been shaped to provide multiple focusing of different frequencies over a selected imaging range in the elevation plane. In particular, high frequencies are focused at shallow ranges and progressively lower frequency components are focused at deeper ranges. The ultrasound system employing the transducer incorporates a time-varying filter (an imaging range dependent filter) in the receiver, which filters the received echo signals to emphasize the frequency components focused at the respective depths.

Multirow transducers (array transducers with several rows of elements) have been developed to obtain better control of the elevation beam profile, enabling dynamic receive processing, but at the expense of increased system complexity (more receive channels, analog-to-digital converters) and cost. Such a transducer is disclosed in e.g. U.S. Pat. No. 5,882,309.

Commonly, the prior art described hereinbefore involve increased complexity either in manufacturing of the transducer elements or in system complexity. Thus, there is a need for a transducer, which provides improved elevation beam performance over that of acoustic single focus lenses well known in the prior art without increasing system and manufacturing complexity and cost.

### SUMMARY OF THE INVENTION

The present invention is an ultrasound transducer with an acoustic lens intended for medical imaging providing continuous focusing in the elevation plane over a predetermined imaging range. The transducer comprises an ultrasound element and an acoustic lens, said lens having a cross sectional profile comprising a curved portion with a preferably circular shape providing a single focus at a first focal depth, and first and second linear portions on either side of said curved portion, said linear portions providing continuous focusing at imaging ranges after said first focal depth of said curved portion. At the interfaces between said curved portion and said linear portions, the slope is continuous such that the surface profile of the lens is smooth. Preferably, said lens is manufactured as a unity. Thereby is obtained a lens surface which is free of abrupt changes not caused by limited accuracy of the manufacturing equipment.

In a first preferred embodiment of the present invention, the acoustic lens is mounted on a conventional 1D ultrasound transducer comprising a plurality of said elements arranged in a single row (and not comprising any prior lens material). Thereby is obtained an ultrasound array transducer which provides a uniform slice thickness throughout the imaging range where the lens provides continuous focusing.

In a second preferred embodiment of the present invention, the acoustic lens is mounted on a single element both of which have preferably a circular shape and circular symmetric geometry. Hereby is obtained a single element ultrasound transducer providing a uniform slice thickness throughout the imaging range where the lens provides continuous focusing which is identical in both the elevation plane and the imaging plane.

### BRIEF DESCRIPTION OF THE DRAWINGS

FIG. 1 is a cross sectional view of a conventional 1D ultrasound transducer comprising an acoustic lens providing a single fixed focus.

FIG. 2A is a cross sectional view of a 1D ultrasound transducer comprising a continuous focusing acoustic lens according to a first preferred embodiment of the present invention.

FIG. 2B illustrates geometric relations applied when designing the invented lens according to the first preferred embodiment.

FIG. 3 shows a contour plot of the elevation beam profile of a conventional 1D ultrasound transducer array comprising a single focus lens such as the one shown in FIG. 1. The level between the contours is 3 dB starting from −3 dB.

FIG. 4 shows a contour plot of the elevation beam profile of a 1D ultrasound transducer array with a continuous focusing lens in accordance with a first preferred embodiment of the present invention, such as the one shown in FIG. 2. The level between the contours is 3 dB starting from −3 dB.

FIG. 5 is a graph showing a comparison of the on-axis pressure field obtained using the single focus lens (dashed) and continuous focusing lens (solid) in the first preferred embodiment.

### DETAILED DESCRIPTION OF THE PREFERRED EMBODIMENTS

The invention is related to the field of medical ultrasound imaging, and in particular, the area of improving the elevation beam profile of the ultrasound transducer to obtain better slice thickness and image contrast resolution by means of extending the depth of focus. The invention is an ultrasound transducer comprising an acoustic element and an acoustic lens. Said element has a substantially uniform frequency amplitude characteristic across its spatial extent and transmitting an ultrasound beam when excited. Said acoustic lens is positioned in front of said element, said lens having a cross sectional profile comprising (1) a curved portion with a curved front surface and a back surface facing said transducer element, said curved lens portion providing a focal point at a first focal range, and (2) a pair of linear portions with linear front surfaces and back surfaces facing said transducer element, said linear portions positioned on either side of said curved portion, and said linear portions providing continuous focusing at imaging ranges after said first focal depth of said curved portion. Said curved portion and said first and second linear portions are combined such that the slope at each interface between said portions is continuous, providing a lens surface which is smooth without any intended abrupt changes. Preferably, the lens is manufactured as a unity.

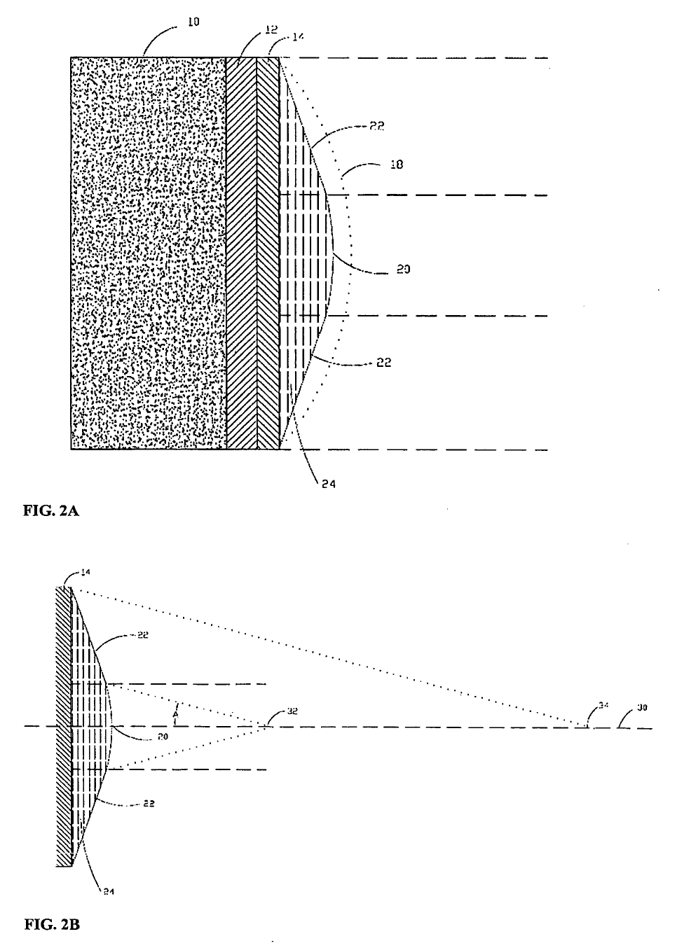

A cross sectional view of an ultrasound transducer according to a first preferred embodiment of the present invention is illustrated in FIG. 2A. The drawing is not drawn to scale and serves only the purpose of illustrating the spirit of this first preferred embodiment of the invention. The element 12, used to convert the electric excitation signal to ultrasound wave in transmission mode and convert received acoustic echoes from the object under investigation (not shown) to electric signals used for generating the displayed image, is made of preferably a piezoelectric ceramic such as PZT. Said element is attached to a backing layer 10, which is introduced to improve the broadband characteristics of the element and attenuate internal reflections, and the matching layer(s) 14 (only one is shown) provide impedance matching to human tissue for more efficient transfer of the acoustic energy. The acoustic lens 24 providing continuous elevation focusing over a selected imaging range according to the invention is connected to the outer matching layer 14 (only one is shown). The lens comprises three portions; a curved portion 20, and two linear portions 22. The curved portion 20 occurs symmetrically around the center of the element, and each linear portion occurs on either side on the curved portion. The cross section of the lens is therefore symmetric about the center axis of the element (not shown here, but shown as line 30 in FIG. 2B). As a reference, the conventional single focus lens is shown in the drawing as the dotted line 18. In this first preferred embodiment, the lens 24 is made of a material having a sound speed which is less than the sound speed of the object to be examined (not shown), said material being preferably a silicone material from the RTV silicone family. Lens materials have in general a high attenuation factor which means that a significant portion of the ultrasound energy produced by the transducer element is absorbed in the lens material and converted to heat. This absorption decreases the ultrasound energy transmitted to the object and is therefore unwanted. The absorption is typically determined by the thickness of the lens material. By means of the invention in this first preferred embodiment, the thickness of the lens material is reduced, resulting in less absorption of ultrasound energy in the lens and an increased amount of energy transmitted to the object, which improves the signal-to-noise ratio and consequently the image quality. The dashed lines 26 indicate the points of transition from said curved portion to said linear portions. In this preferred embodiment, the curved portion 20 has a circular front surface facing the object to be examined (not shown) providing a single focus at a first focal range. Each linear portion 22 has a slope which is equal to the slope at the edge of the circular portion corresponding to the point, where the respective linear portion and the circular portion connect. This produces a lens surface with a continuous slope across the entire profile (neglecting any inaccuracy related to the manufacturing of the lens). This is an essential characteristic of the invented lens, because abrupt changes in the lens profile may introduce unwanted effects in the elevation beam profile, affecting the quality of the resulting images. The linear portions 22 provide continuous focusing at imaging ranges after the first focus point of said circular portion. The continuous focusing effect is obtained by means of the linearly decreasing lens thickness and the lower sound speed of the lens material compared to the typical sound speed of the object to be examined. In particular, the continuous focusing at imaging ranges after the first focal depth is ensured by maintaining the slope at the edges of the curved portion in the respective linear portions. The substantially uniform frequency amplitude characteristic of said transducer element is obtained by means of a substantially uniform cross sectional thickness over the spatial extend of the element, because the resonance frequency of the element is inversely proportional to the element thickness. In this first preferred embodiment, the substantially uniform cross sectional thickness is obtained by means of a rectangular cross section of the element 12 as illustrated in FIG. 2A. Together with the application of the backing layer 10, the transducer transmits and receives broadband ultrasound waves at each point on the surface and in accordance with an objective of the present invention maintains the broadband characteristic of the transducer and focused all frequencies at all imaging ranges where the lens provides continuous focusing. This makes this preferred embodiment particularly useful for harmonic ultrasound imaging, where higher order frequency components, most commonly the second harmonic component, are used for generating the ultrasound images. To enable this, a transducer with a high frequency bandwidth (broadband) is required.

FIG. 2B is a drawing illustrating the geometric setup used to design the lens according to the first preferred embodiment of the invention described hereinbefore. As for FIG. 2A, the drawing serves only the purpose of illustration and is not drawn to scale. The figure shows a cross sectional view of a transducer according to the invention, similar to the transducer in FIG. 2A, where only the outer part of the matching layer 14 and the acoustic lens 24 comprising a curved portion 20 and two linear portions 22 is shown. The location of said first focal point 32 produced by the circular surface curvature of the curved portion in this preferred embodiment is determined by the radius 36 of said circular curvature, the sound speed of the lens material, and the typical sound speed of the intended objects to be examined. The spatial extent of the circular portion is determined by angle A, which is measured as the angle between the symmetry axis 30 and the line extending from the first focal point 32 to the upper edge point of the circular portion 20 indicated by the upper dashed line 26, as illustrated in the drawing. The linear portions 22 provide continuous focusing at imaging ranges between said first focal depth 32 and the last focal point 34. The continuous focusing effect is obtained by means of the linearly decreasing lens thickness and the lower sound speed of the lens material of this preferred embodiment compared to the typical sound speed of the object to be examined, preferably human tissue. In particular, said continuous focusing is ensured by maintaining the slope at the edges of the curved portion 20 in the respective linear portions 22.

In one aspect of the first preferred embodiment described hereinbefore, the transducer comprises a plurality of said element preferably arranged along a single row, producing a 1 D transducer array. Typically, said transducer comprises 128 to 256 of said elements. Said elements having preferably a height (the elevation aperture size) which is considerably larger than the width (the azimuth or lateral element size). Said plurality of elements are displaced uniformly across the array with an element-to-element distance (often denoted the pitch), which is typically less than the ultrasound wavelength, depending on the intended application of the probe. In a preferred embodiment, the height is approximately 13 mm. In an alternative embodiment, the element height is in the range 13 mm+/−1 mm. In another alternative embodiment, the element height is in the range 10 mm+/−2 mm. In yet another alternative embodiment, the element height is in the range 6 mm+/−2 mm. In a preferred embodiment, the pitch is approximately equal to the wavelength of the transmitted ultrasound beam. In another alternative embodiment, the pitch is in the range [wavelength; wavelength-5%]. In yet another alternative embodiment, the pitch is in the range [wavelength-5%; wavelength-10%]. In yet another alternative embodiment, the pitch is in the range [wavelength-10%; wavelength-15%]. In a preferred embodiment, the angular size A of the curved portion is approximately 2°. In another preferred embodiment, the angular size A of the curved portion is 2°+/−0.2°. In another preferred embodiment, the angular size A of the curved portion is 2°+/0.4°. In another preferred embodiment, the angular size A of the curved portion is 2°+/0.6°. In another preferred embodiment, the angular size A of the curved portion is 2°+/0.8°. In another preferred embodiment, the angular size A of the curved portion is 2°+/−1°. In yet another preferred embodiment, the angular size A of the curved portion is 3.5°+/−0.5°.

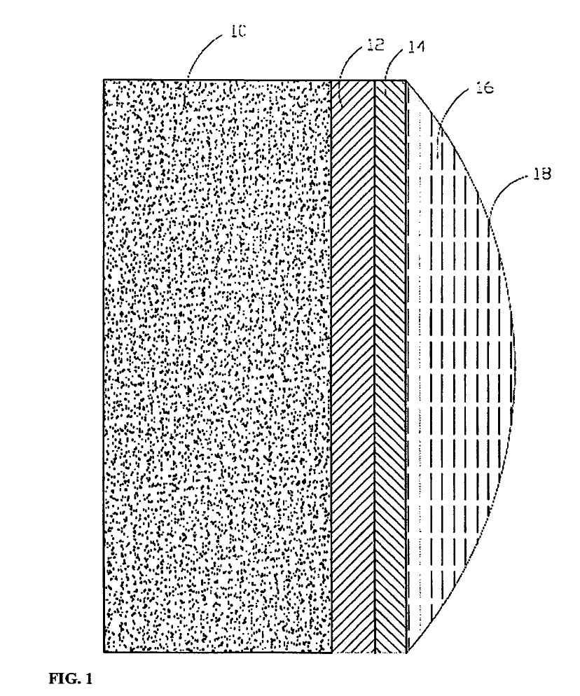

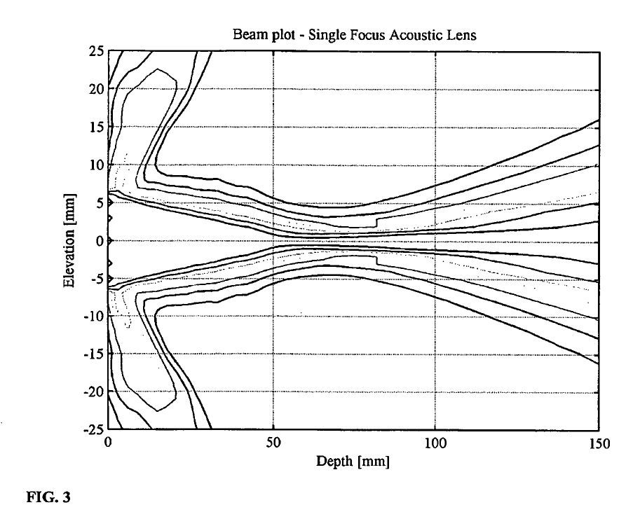

FIG. 3 shows a contour plot of the elevation beam profile for a conventional 1D ultrasonic transducer array comprising a single focus lens such as the one shown in FIG. 1. The elevation aperture, i.e. the height of each element, is chosen to 13 mm, and the lens provides a focal point at 65 mm. The level between the contours is 3 dB. It is noticed, that the beam diverges after the focal point producing a non-uniform beam profile which leads to degraded contrast resolution performance at deeper imaging ranges, significantly at ranges after 100 mm.

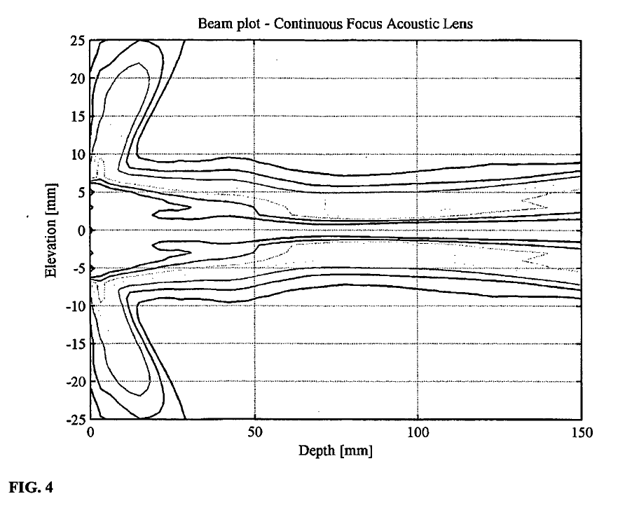

FIG. 4 shows a contour plot of the elevation beam profile for at 1D ultrasound transducer comprising a continuous focusing lens in accordance with a preferred embodiment of the present invention, such as the one shown in FIG. 2A. The elevation aperture is 13 mm. The curved portion of the lens provides a single focus at 40 mm and the outer segments provides continuous focusing at imaging ranges from 40 mm to approximately 180 mm. The angular size A of the curved portion is 2° measured from the 40 mm focal point 32 as illustrated in FIG. 2B, corresponding to an elevation aperture of 2.8 mm. In accordance with one objective of the present invention, the beamwidth produced by the lens is uniform throughout the majority of the imaging range. In accordance with another objective of the present invention, all transmitted frequencies are focused at all imaging depths within the focal length, maintaining the desired broadband characteristic of the invented transducer.

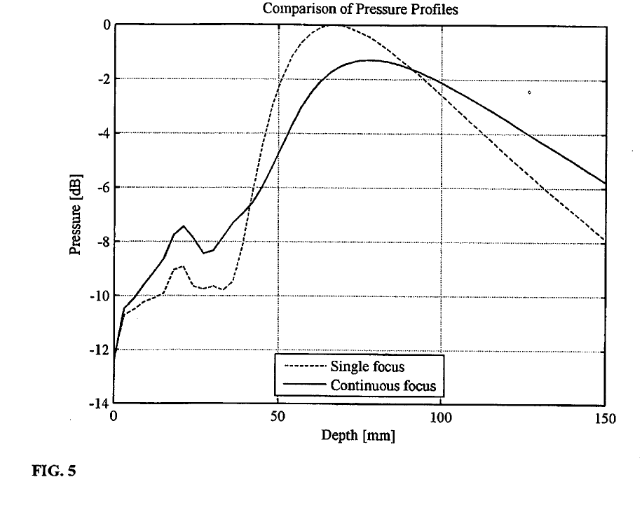

In FIG. 5 the on-axis pressure profiles of a single focus lens (dashed) and a continuous focusing lens (solid) according to the preferred embodiment are compared. Several advantages of the continuous focusing lens pressure field are observed. The peak pressure at the acoustical focal point has been decreased, correspondingly reducing the mechanical index (MI). Furthermore, the acoustic pressure has been increased in the near and far field, improving the signal to noise ratio of the received echoes from the respective ranges. Due to the decrease in peak pressure, the transmitted power can be increased accordingly, resulting in a positive effect on the signal to noise ratio and hence image quality.

In a second aspect of the first preferred embodiment, the transducer comprises a single of said element, which is moved, preferably rotated, mechanically to obtain sector images, and the continuous focusing acoustic lens described hereinbefore. At multiple spatial positions an ultrasound beam is transmitted and the reflected echoes from the object are processed and displayed. Said element has a preferably circular shape and is circular symmetric. Said lens has a preferably circular shape and is circular symmetric to match the circular element. The circular geometry of said element and lens provides a beam profile which is identical in both the elevation plane and the imaging plane. Therefore, a uniform beam width and hence slice thickness is obtained, enhancing the transducer performance compared to conventional single element transducers comprising a single focus.

The preferred embodiments of the present invention described hereinbefore serve the purpose of illustration of the invention. Numerous variations and modification are readily apparent within the spirit of the present invention to those skilled in the art of developing medical ultrasound transducers. All such variations and modifications an intended to be encompassed by the following claims:

### REFERENCES

1. Min-Kang Chao and Sheng-Wen Cheng: Aspheric Lens Design. Proceedings of the 2000 IEEE Ultrasonics Symposium
2. Umemura, S.-i.; Azuma, T.; Miwa, Y.; Sasaki, K.; Sugiyama, T.; Hayashi, T.; Kuribara, H.: Non-Cylindrical Transmission Focusing for Large Depth of Field, Proceedings of the 2002 IEEE Ultrasonics Symposium

### CITED PATENTS

1. U.S. Pat. No. 5,083,568
2. U.S. Pat. No. 5,678,554
3. U.S. Pat. No. 5,882,309
4. U.S. Pat. No. 6,027,448

What is claimed is:

1. An ultrasound transducer with an acoustic lens, said lens having a cross-sectional profile through which ultrasound is transmitted by means of an acoustic element with a spatial extend;
a. Where said acoustic element has a frequency amplitude response which is substantially uniform across the spatial extend;
b. Where said profile comprises a first linear portion, a curved portion, and a second linear portion;
c. Where said acoustic lens has said profile as its front surface to provide continuous focusing over a predetermined range.

2. An ultrasound transducer with an acoustic lens according to claim 1, where the slope is continuous over said profile.

3. An ultrasound transducer with an acoustic lens according to claim 1, where said lens is manufactured as a unity.

4. An ultrasound transducer with an acoustic lens according to claim 1, said transducer comprising a plurality of said element.

5. An ultrasound transducer with an acoustic lens according to claim 3, where said elements are arranged along a single row.

6. An ultrasound transducer with an acoustic lens according to claim 1, said transducer comprising a single of said element.

7. An ultrasound transducer with an acoustic lens according to claim 1, where said element is made of piezoelectric ceramic.

8. An ultrasound transducer with an acoustic lens according to claim 1, where said acoustic lens is made of a silicone material from the RTV silicone family.
